SEQUENCE LISTING (2 sequences)

SEQ ID NO: 1 - protein, 256 aa, Pyrococcus
MIFKAKFGEPKRGWVVIVHGLGEHSGRYAKLVEMLVERGFAVYTFDWPGHGKSSGKRGHTSVEEAMEIIDEIIEEIGEKPFLFGHSLGGLTVIRYAETRPEKVKGVIASSPALAKSPNTPGFLVALAKFLGVVAPGITFSNGINPNLLSRNKDAVRRYVEDPLVHDKITAKLGRSIFMNMELAHREAEKIKVPILLLVGTQDVITPPEGARKLFEKLKVEDKEIREFEGAYHEIFEDPEWGEEFHRVIVEWLEKHS

SEQ ID NO: 2 - protein, 260 aa, Thermococcus
MEVYKVRFGTPERGWVVLVHGLGEHSGRYGRLIKLLNENGFGVYAFDWPGHGKSPGKRGHTSVEGAMEIIDSIIEELGENPFLFGHSLGGLTVIRYAEARPDKIRGVIASSPALAKSPETPDFMVALAKFLGRIAPGLTLSNGIKPELLSRNRDAVRRYVEDPLVHDRISAKLGRSIFVNMDLAHREAENIRVPILLLVGTGDVITPPKGAKDLFKKLKVEDKELKEFPGAYHEIFEDPEWGEEFHKTIVEWLLQHSEEG

(19) United States
(12) Patent Application Publication
(10) Pub. No.: US 2006/0008887 A1
(43) Pub. Date: Jan. 12, 2006

(54) Production of carboxylic acid and carbonic acid derivatives using a thermostable esterase

(76) Inventors: Burghard Gruning, Essen (DE); Geoffrey Hills, Essen (DE); Thomas Veit, Hagen (DE); Christian Weitemeyer, Essen (DE); Olivier Favre-Bulle, Nimes (FR); Fabrice Lefevre, Nimes (FR); Hong-Khanh Nguyen, Nimes (FR); Gilles Ravot, Nimes (FR)

Correspondence Address: SCULLY SCOTT MURPHY & PRESSER, PC, 400 GARDEN CITY PLAZA, SUITE 300, GARDEN CITY, NY, 11530, US

(21) Appl. No.: 10/955,053

(22) Filed: Sep. 30, 2004

(30) Foreign Application Priority Data
Sep. 30, 2003 (EP) 03021973.7

(51) Int. Cl. C12P 7/64

(52) U.S. Cl. 435/134

(57) ABSTRACT

The present invention relates to processes for the production of acyl compounds using an esterase having thermostable properties, and to products of such processes.

### FIELD OF THE INVENTION

The present invention relates to processes for the production of acyl compounds using an esterase having thermostable properties, and to products of the inventive processes.

### BACKGROUND OF THE INVENTION

The use of biological catalysts for the production of acyl compounds has several advantages, such as, for example:
the desired products can be efficiently produced, based on the substrate specificity and site specificity of the enzyme.
by-products which are frequently produced in chemical reactions are avoided, thus also avoiding costly and time-consuming purification measures.
because of lower temperatures, as in conventional processes, energy for heating and cooling can be saved.

The products obtainable by the above reactions often are oleochemically derived with some more or less pronounced surface or interfacial activity. The products are widely used in various applications, including, for example, in food and feed, cosmetics and toiletries, pharmaceuticals, agriculture, and other various technical applications.

There are numerous raw materials and products in the field of oleochemistry and surfactant chemistry which melt at temperatures of about 65° C. or higher, e.g., stearic acid at 71° C. and behenic acid at 79° C. To carry out enzymatic reactions without using solvents, which are generally undesired and also will increase production costs considerably, enzymes are needed which are stable well above 60° C. Another advantage of performing reactions at elevated temperatures is the viscosity reducing effect. This is especially desired if oligomers or polymers are converted by enzymatic reactions.

Another aspect to highlight the advantages of thermostable enzymes, which is especially important in the synthesis of surface active compounds, is the reaction of hydrophilic and lipophilic compounds which need to be compatibilized to react with each other. For this purpose, a simple and efficient means is to increase the temperature of the processes, which again requires suitable enzymes.

Esterases belong to the class of hydrolases. The hydrolases are widely used as biocatalysts having an enzymatic activity of esterases, lipases, phospholipases, lysophospholipases or amidases for carrying out hydrolysis reactions, acidolysis reactions, or transformation reactions like trans-esterification. As used in the present application, the term esterase encompasses lipases, phospholipases, lyso-phospholipases, or amidases.

Industrially used esterases have been isolated from a broad variety of organisms, including bacteria, yeast, higher animals and plants. However, most esterases have a limited operational temperature range, and are not suited for operations in the pharmaceutical or oleochemical industry many which have to be conducted at increased temperatures.

Products for the mentioned application areas like cosmetic and toiletries, pharmaceuticals, and various technical applications contain, as raw materials besides some basic compounds, other highly efficient ingredients to achieve specific effects. These ingredients necessarily are of a pronounced speciality character and often need to be designed specifically for the desired application. Thus, many different products which are used as raw materials need to be provided and produced economically to fulfil all the specific requirements in the application areas. Versatile multipurpose facilities and processes are needed to produce quality products. Although enzymatic processes have many advantages, such processes are often too specific and too sensitive to offer an economical production route. Therefore, there is a desire for new enzymes which are robust and can be used in a flexible way. The enzyme must be useable at increased temperatures and active on a broad range of substrates, such as, short and long chain alkyl residues. The enzyme must be able to catalyse processes wherein oil as well as water soluble raw materials are used or products made.

In the last two decades, the discovery and isolation of thermophilic bacteria, such as eubacteria or archaea, isolated from, e.g., hot springs, “black smokers” or deep-sea hydrothermal vents, has resulted in the identification of new hydrolases which function at temperatures above 60° C., where most other proteins are deactivated.

Esterases and lipases can be characterized by different substrate specificities, substituent group or chain length preferences, and unique inhibitors. See, for example, Barman, T. E. Enzyme Handbook, Springer-Verlag, Berlin-Heidelberg, 1969; Dixon, M. et al. Enzymes, Academic Press, New York, 1979. Esterases are able to carry out reactions, i.e., the hydrolysis of ester bonds in aqueous and organic solvents. The major activity of these enzymes is the hydrolysis of ester bonds to carry out reactions on a wide variety of substrates, including esters containing cyclic and acyclic alcohols, mono- and di-esters, and lactams. See Santaniello, E., et al., The biocatalytic approach to the preparation of enantiomerically pure chiral building blocks, Chem. Rev. 92:1071-1140, 1992. Esterases can catalyze esterification or acylation reactions to form ester bonds (Santaniello, E. et al., supra). This process can also be used in the transesterification of esters, and in ring closure or opening reactions.

Esterases are a group of key enzymes in the metabolism of fats and are found in all organisms from microbes to mammals. In the hydrolysis reaction, an ester group is hydrolyzed to an organic acid and an alcohol.

Industrial and scientific applications for esterases are:
1) Esterases in the dairy industry as ripening starters;
2) Esterases in the pulp and paper industry for lignin removal from cellulose pulps, for lignin solubilization by cleaving the ester linkages between aromatic acids and lignin and between lignin and hemicelluloses, and for disruption of cell wall structures when used in combination with xylanase and other xylan-degrading enzymes in biopulping and biobleaching of pulps;
3) Esterases in the synthesis of carbohydrate derivatives, such as sugar derivatives;
4) Esterases in combination with xylanases and cellulases, in the conversion of lignocellulosic wastes to fermentable sugars for producing a variety of chemicals and fuels;
5) Esterases as research reagents in studies on plant cell wall structure, particularly the nature of covalent bonds between lignin and carbohydrate polymers in the cell wall matrix;
6) Esterases as research reagents in studies on mechanisms related to disease resistance in plants and the process of organic matter decomposition;
7) Esterases in selection of plants bred for production of highly digestible animal feeds, particularly for ruminant animals;
8) lipases in the hydrolysis of fats and oils to produce fatty acids;
9) Lipases in the transesterification of fats and oils to produce special fits.

Most of the current processes for the production of carboxylic acid and carbonic acid derivatives use esterases which are not robust and not adequate in stability against elevated temperatures, and are therefore not practical for industrial applications at increased temperatures, broad pH-value ranges, on different kind of substrates like short and long alkyl residues, and in various media, such as organic solvents, or they are not suited for long-term reactions.

U.S. Pat. No. 5,604,119 describes a process for producing triglycerides from glycerol with a long-chain polyunsaturated fatty acid having at least 20 carbon atoms and at least 3 double bonds or a C₁₋₄ alkyl ester thereof using an immobilized lipase from Candida Antarctica, which is thermostable for 24-48 h with a temperature optimum of 40-80° C. The examples in the '119 patent only disclose reactions conducted at 65° C. The immobilized lipase could be reused under the same conditions without excessive loss of activity. The '119 patent does not disclose the use of the lipase for other purposes than the preparation of triglyceride from a polyunsaturated fatty acid having at least 20 carbon atoms and at least 3 double bonds, or a C₁₋₄ alkyl ester thereof.

U.S. Pat. No. 5,480,787 discloses a transesterification method of carboxylic acid esters and alcohols using a lipase powder, preferably with a pulverized commercially available lipase from Alcaligenes which is used at temperatures between 81-130° C. for 10 min to 50 h for the transesterification of oils, fats and resins. In the '787 patent it is indispensable that the lipase is added directly to the ester to be dispersed, and not to the carboxylic acid or the alcohol as the enzyme looses activity therein. Preferably, the enzyme is solubilized in an inert organic solvent. The dispersed enzyme-substrate solution has to be homogenized thereafter by ultrasonic treatment of the inert organic solvent and/or ester containing the lipase powder. Alternatively, the dispersion is stirred, and then subjected to microfiltration and centrifugal precipitation to obtain a dispersion wherein at least 90% of the lipase particles have a diameter in the range of 1 to 100 μm. Furthermore, this process is less efficient when the amount of the dispersed particles with a diameter of 1-100 μm is below 90%, as the esterase activity is reduced (if the particle diameter is larger), and the recovery of the lipase particles from the reaction liquid is difficult to make or the reuse thereof impossible (if the diameter is smaller). For an optimal conduction of the process, and as the lipase is not immobilized on a carrier, the particle diameter has to be controlled in the course or after the completion of the reaction which makes the process costly, and laborious. Furthermore, no esterification or hydrolysis reactions or any reactions involving amine compounds are disclosed, and none of the examples in the '787 patent discloses the use of an organic solvent, and additionally there is no indication of the pH-range at which the enzyme may be used. According to said the '757 patent, the immobilizate of enzymes in transesterification methods is disadvantageous as the lipase activity would be reduced, and side reactions would be caused by the introduction of water into the immobilizate carrier. Moreover, the Alcaligenes lipase itself is not thermostable, as is shown in example 18 of the present application

EP 0 709 465 and EP 0 714 984 describe a process for the production of optically active alcohols by interesterification between a racemic alcohol and an ester with a thermostable lipase derived from Alcaligenes under water-free conditions, and at temperatures between 81° C. to 120° C. The lipase can either be immobilized on a carrier or used in powdered form. The particle diameter has to be controlled strictly. Thus, the process disclosed in the aforementioned European Applications suffers from the disadvantage, that a dispersion step is necessary before carrying out the enzymatic reaction Both European Applications do not disclose the use of said lipase for hydrolysis or esterification reactions. Additionally, none of the examples provided in the aforementioned European Applications discloses the use of an immobilized enzyme. These European Applications also do not disclose, whether the enzyme is reusable. Moreover, the Alcaligenes lipase itself is not thermostable, as is shown in example 17 of the present application

U.S. Pat. No. 5,273,898 describes a process for the hydrolysis, synthesis or interesterification of an ester by two lipase fractions derived from C. Antarctica. One of these fractions is more temperature-stable, the other more pH-stable. Temperatures of these reactions are 60-90° C., preferred 60-80° C., however, the temperature optimum is 65° C., and the examples provided in the '898 patent do not disclose reaction temperatures above 90° C. Additionally, the enzyme of the invention is an immobilized enzyme. Thermostability of the enzyme itself is only shown for 30 min at 84° C. at maximum.

Hotta, et al. (Appl. Environ. Microbiol. 68, pp. 3925-3931, 2002) found and characterized a thermostable esterase in the archaeon Pyrobaculum calidifontis. The esterase was shown to be thermostable for at least 2 h at 100° C. and to have a half-life-time at 110° C. of 56 min, both measured in aqueous medium. The esterase is also well stable in the presence of water-miscible organic solvents. Its' substrate specificity is limited to short hydrocarbon chain substrates with an optimum for C₆.

In the light of the prior art mentioned above, it is desirable to provide efficient and versatile processes for the production of acyl compounds which can be conducted for a prolonged period of time at elevated temperatures, with a large variety of substrates of different structures, of low and high molecular weight, with various carbon chain lengths, in various media and, if water is present, in a broad pH-range.

### SUMMARY OF THE INVENTION

The present invention relates to a process for the production of acyl compounds of the general formula R¹[(—X—R²)ₙR³]ₚ wherein R¹ is hydrogen or an organic or silicone-organic residue which can be cyclicly connected to R⁶, X is C(O)—Y, Y—C(O), C(O)—R⁴—C(O)—Y, or Y—C(O)—R⁴—C(O), R² is a group of divalent organic residues containing p members from R^{2.1} to R^{2.p} which can be equal or different, each containing at least one carbon atom, R³ is a chemical univalent link or selected from the group of hydrogen, an hydroxyl group, an alkyl group which can be cyclicly connected to R⁵, the group Y—C(O)—R⁴H, or Y—C(O)—R⁵—C(O)—OH, n is an integer number ≧1, p is an integer number from 1 to 100, Y is O, NR⁶, or S, R⁴ is a divalent hydrocarbon group which can be saturated or unsaturated, linear, branched, or cyclic, or a silicone-organic group, R⁵ is a divalent hydrocarbon group which can be saturated or unsaturated, linear, branched or cyclic, not substituted or substituted by hydroxy, alkoxy, hydroxycarbonyl or alkoxycarbonyl groups, R⁶ is hydrogen, a mono or divalent hydrocarbon group, which can be saturated or unsaturated, linear, branched, or cyclic, not substituted or substituted by hydroxy or alkoxy groups, and cyclicly connected to R¹ or R³, by contacting an immobilized thermostable esterase with carboxylic acid derivatives and water, alcohols, amines, or thiols for hydrolysis or the formation of esters amides, or thioesters. In accordance with the present invention, the esterase
a) retains, in its' free form, at least 10% of its' initial hydrolysis activity after treatment for 40 h at 80° C. in aqueous solution,
b) has an optimal temperature of 70 to 110° C., and
c) is suitable for repeated use in the process at temperature above 70° C.

Preferably, the esterase retains, in its' free form, at least 10% of its initial hydrolysis activity after treatment for 40 h at 90° C., most preferable at 100° C. in aqueous solution.

### BRIEF DESCRIPTION OF THE DRAWINGS

FIG. 1 is a map of the transformation vector pARA P 1021.

FIG. 2 is a graph of conversion vs. time which determines the esterification activity of immobilized recombinant Est P 1021.

### DETAILED DESCRIPTION OF THE INVENTION

The present invention relates to a process for the production of compounds of the general formula
R¹[(—X—R²)ₙR³)]ₚ (I)
using the above mentioned enzyme.

In formula (i), R¹ is hydrogen or an organic or silicone-organic residue which can be cyclicly connected to R⁶. Preferably, R¹ is an alkyl, alkenyl, alkylen or alkenylen group or a silicone-organic group, which can be substituted by hydroxyl or amino groups and which can be interrupted by oxy-groups. R¹ is typically derived from an alcohol, amino or thiol compound of the formula R¹OH, R¹NR⁶H, or R¹SH, or their acyl derivatives like esters or amides, preferably R¹OH or R¹NH₂. R¹ may also be part of a carboxylic acid derivative. R¹ preferably is derived from an alcohol, which can be mono- or polyvalent. A few examples are decanol, benzylalcohol, glycerol or sorbitol. X is C(O)—Y, Y—C(O), C(O)—R⁴—C(O)—Y or Y—C(O)—R⁴—C(O), wherein Y is O, NR⁶, or S, R⁴ is a divalent hydrocarbon group which can be saturated or unsaturated, linear, branched, or cyclic or a silicone-organic group, and R⁶ is hydrogen, a mono or divalent hydrocarbon group, which can be saturated or unsaturated, linear, branched, or cyclic, not substituted or substituted by hydroxy or alkoxy groups. If R⁶ is different from hydrogen, it can be cyclicly connected to R¹ or R³. The group X is typically an ester, amide or thioester group, preferentially an ester or amide group and especially preferred an ester group.

Another preferred embodiment is that R⁴ connects two carboxyl or carboxyl-derived groups. R⁴ might also be a divalent silicone-organic residue. Thus, the total group is derived from a dicarboxylic acid. R² is a group of a divalent organic residues containing p members from R^{2.1} to R^{2.p} which can be equal or different, each containing at least one carbon atom. Preferably, R² is a divalent hydrocarbon group, which can be saturated or unsaturated, linear, branched, or cyclic, and which can be interrupted by ether groups. More preferred R² groups are alkylen or alkenylen groups with 5 to 50 carbon atoms.

A compound of the general formula (I) may contain several different groups R². This fact is basically related to the functionality of R¹, if it is >1 and carries p residues the compound may contain p different residues. Especially preferred groups R² are derived from fatty acids containing 8 to 30 carbon atoms. R³ is a chemical univalent link hydrogen, an hydroxyl group, an alkyl group which can be cyclicly connected to R⁵, the group Y—C(O)—R⁴H, or Y—C(O)—R—C(O)—OH. Preferably, R³ is hydrogen or an alkyl group. The index n is an integer number ≧1 and it represents the degree of polycondensation, n is preferably in the range of 1 to 10.000, more preferably 1 to 500. The index p is an integer number from 1 to 100. The index p reflects the functionality of R¹ which is ≧p, and it represents the number of residues (—X—R²)ₙR³ bound to R¹. The index p is preferably in the range of 1 to 30 and more preferably in the range of 1 to 8. The following examples are provided to understand the nature of the chemical structures of the compounds of the general formula (I.):

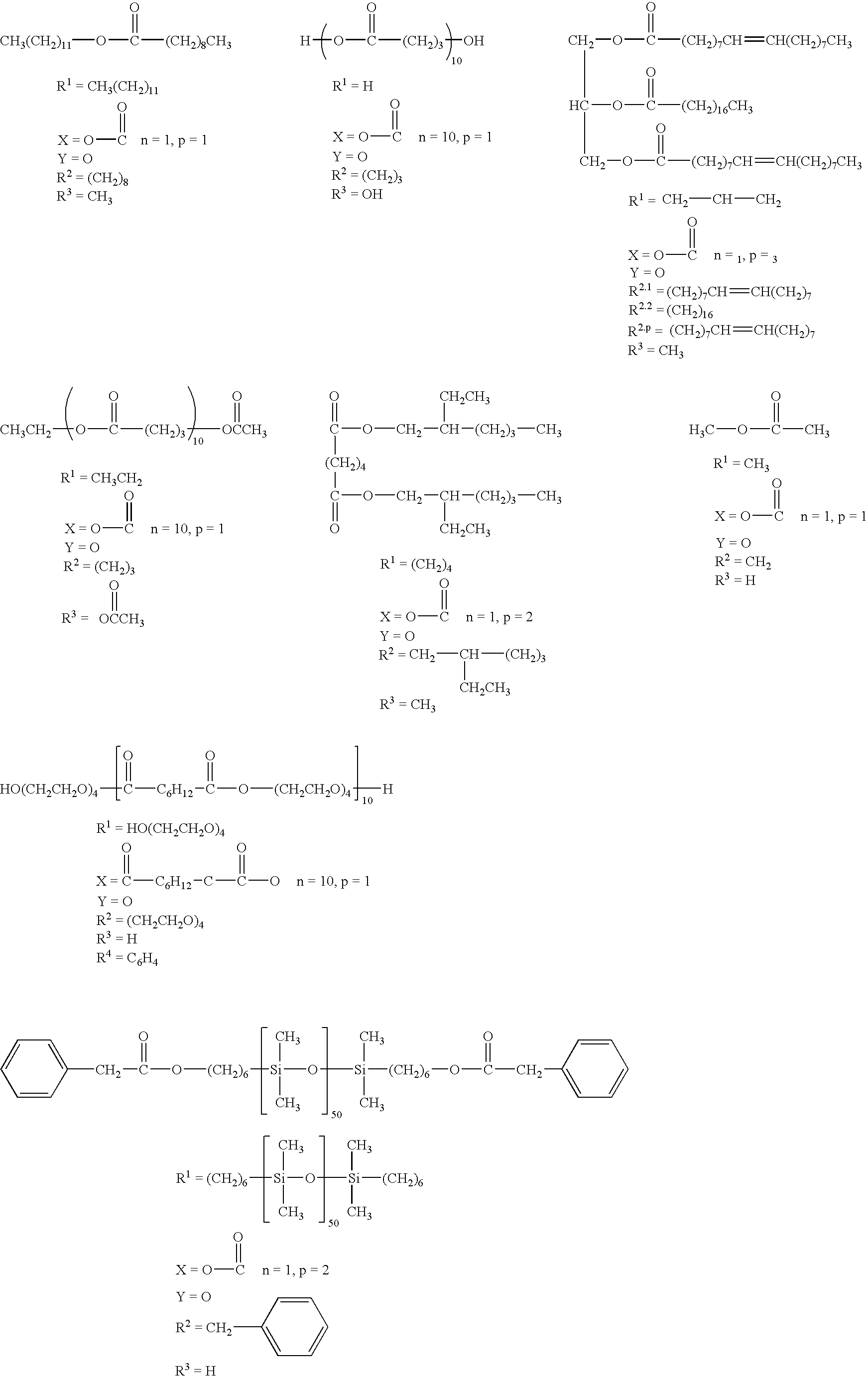

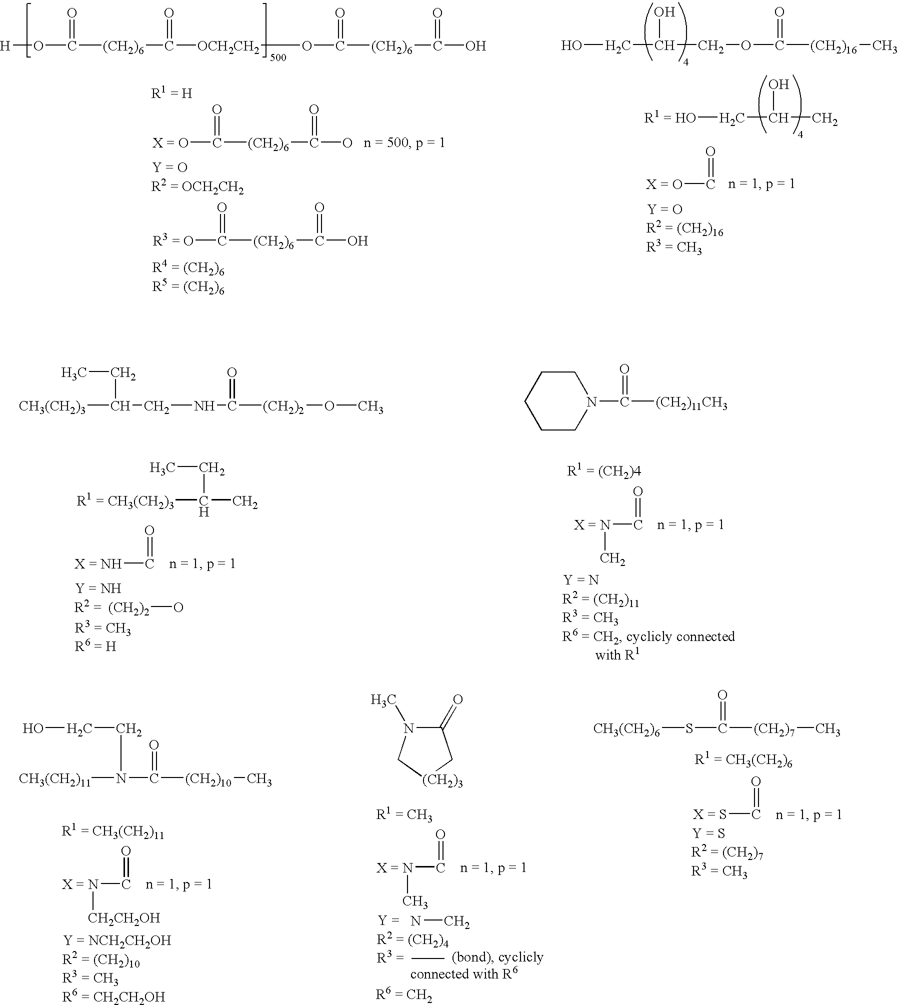

In the inventive process, the enzyme is suitable to catalyze the formation of esters, amides, or thioesters or their hydrolysis. The esters, amides, or thioesters may be formed by an esterification or amidation reaction starting from the corresponding alcohols, amines, thioalcohols and carboxylic acids or by transformation of esters and amides with alcohols, thioalcohols, or amines. Another type of reaction to form the compounds of formula (I), which is also within the scope of the present invention is the conversion of two esters in an interesterification reaction.

Examples of acyl compounds, which are used in the inventive process for the production of compounds of the general formula (I) can be derived from carbonic acid and carboxylic acids like acetic, butyric, caprylic, isononanoic, isostearic, oleic, 4-hydroxybutyric, 12-hydroxystearic, 2-ethylhexanoic, cyclohexylacetic, malic, adipic acid. They further can be terpenoid like cholic acid, sugar derived like glucuronic acid or amino acid derived like N-acetylalanin. Examples of polymeric acyl residues are polyacrylic acid or carboxyfunctional silicone compounds which can be made by the addition of undecylenic acid derivatives to functional silicones (see, for instance, U.S. Pat. No. 4,725,658).

Examples of alcohol components which can be used in the inventive process are methanol, isopropanol butanol behenyl alcohol, octacosanol, Unilin™ 425 (Baker Petrolite, Baker Hughes Inc.), 2-ethyl-1,3-hexanediol isononanol, isostearyl alcohol trimethylolpropane, 3-phenoxypropanol, retinol,1-phenylethanol, ethyleneglycol glycerol ascorbic acid, α-methylglucoside, sorbitol, 4-hydroxybutyric acid, epilupinine, 2-2-aminoethylamine ethanol, dipropylene glycol, polyglycerol, polyethylene glycol, polyvinyl alcohol, hydroxyfunctional silicones like they can be obtained by the addition of 1-hexen-6-ol to Si—H functional silicones or as described, e.g., in U.S. Pat. No. 2,924,588 or in DE-OS-40 10 153.

Examples for amino components which can be used in the inventive process are butylamine, stearylamine,3-dimethylaminopropylamine, 2-2-aminoethylamine ethanol diethylenetriamine, 2-ethylhexylalanin and an example for a thiol component is lauryl mercaptan.

Preferably, the substrates of the present invention can be monomeric or polymeric, of low or high molecular weight from 10 to 100,000. Besides C, H, N, and O, the substrates can further contain Si, P, or S. Furthermore, they can be organic or silicone organic. The organic residues can be interrupted by oxy-, imino-, or thiogroups being ether or thioether residues or secondary or tertiary amine residues.

Especially preferred acid components used for the production of compounds of the general formula (I) are fat or oil derived or silicone derivatives.

Especially preferred alcohol components are aliphatic alcohols, glycerine or sugar derived alcohols or silicone derivatives.

More preferably, the substrates used in the process of the present invention for the production of compounds of the general formula (I) have chain lengths from 2-50, preferably 2-24, more preferably 3-18 carbon atoms.

In another preferred embodiment of the present invention, one of the substrates for the production of compounds of the general formula (I) is polar and the other is nonpolar. The polar substrate may be water soluble, while the nonpolar substrate may be oil soluble.

The reaction media in which the processes of the present invention using a thermostable esterase for the synthesis of esters, amides or other acyl compounds are conducted, consists of alcohols and carboxylic acids, alcohols and esters, carboxylic acids and esters, esters and alkylamines to mention some examples. The reaction media may additionally contain polar or non-polar solvents, such as t-butanol, dioxane, decalin, or petrolether.

The esterase used in the present invention retains, in its' free form, at least 10% of its' initial hydrolysis activity after treatment for 40 h at 100° C. in aqueous solution, has an optima temperature of 70 to 110° C. and is suitable for repeated use in said process at temperatures above 70° C. The hydrolysis activity of the esterase can be determined by the hydrolysis of 2-hydroxy-4-p-nitrophenoxybutyl decanoate.

The esterase used in the present invention can further be characterized by a temperature optimum of from 70 to 110° C., preferably 80 to 105° C., most preferably 90 to 100° C., an enzymatic activity in a pH range of pH 3-pH 9, and a pH optimum at pH 3-pH 7. The esterase can also be used as a lipase, phospholipase, or lysophospholipase, preferably as a lipase.

The esterase of this process is suitable for repeated use in the inventive process. For this purpose, the esterase is immobilized which can be achieved by techniques which are well known to those skilled in the art.

There are numerous techniques known for immobilization. Usually, such techniques involve the attachment of the enzyme onto a solid support by adsorptive means or also by covalent binding. There are also other techniques which use cross-linking of the enzyme in free or in crystalline form. Also, confining the enzyme into a restricted area, like entrapment into a solid matrix or a membrane-restricted compartment, is possible and is frequently used. Depending on the immobilization technique, the properties of the biocatalyst such as, stability, selectivity, binding properties for substrates, pH and temperature characteristics, can be changed.

Cross-linking of enzymes means the attachment of enzyme molecules with other enzyme molecules by covalent bonds. See, for example, (S. S. Wong, L-JC. Wong Enzyme Microb. Technol. 14 (1992) 866) which results in insoluble high-molecular aggregates. The free enzyme molecules can also be cross-linked with other inactive “filler” proteins such as albumins. The most widely used reagent for immobilization by cross-linindg is α,ω-glutardialdehyde (S. S. Khan, A. M. Siddiqui Biotechnol. Bioeng. 27 (1985) 415), sometimes in combination with other cross-linkers like polyazetidine. The advantage of this method is its simplicity.

The esterase can be recovered by filtration, centrifugation, or any other means and used again in the next batch. The esterase can also be part of a packed bed reactor and thus be used repeatedly. The esterase should be usable at least 3 times without loosing more than 80% of its initial activity, preferably the esterase should be reusable at least 10 times even more preferably 30 times in said process.

Additionally, the process according to the present invention includes an esterase which maintains its activity in a range of solvent conditions, including aqueous, polar and non-polar media. More particularly, the processes involve an esterase which has a residual activity of at least 30% after heat treatment at 90° C. for 30 min, preferably at least 40%, most preferably at least 50% in aqueous, polar or non-polar organic media like alcohols, ketones, esters, carboxylic acids, aliphatic and aromatic hydrocarbons. Examples are ethanol isopropanol t-butanol, ethyleneglycol, acetone, cyclohexane, ethyleneglycol dihydroxystearate, methylcyclohexane and toluene.

Preferred esterases are characterised by an activity index a ≧0.02, wherein a=b·c and b is the fraction of relative activity in the hydrolysis at 80° C. and 40 min of 2-hydroxy-4-p-nitrophenoxy-butyl decanoate after versus before treatment of the enzyme for 40 h at 100° C. in aqueous solution and c is the fraction of relative activity in the transesterification reaction: methyl laurate+decanol→decyl laurate+methanol at 80° C. and 24 h after versus before treatment of the enzyme for 24 h at 80° C. in methylcyclohexane.

The index ‘a’ is an expression of the thermostability of said ester in hydrolysis and synthesis reaction conditions. The numbers ‘b’ and ‘c’ are relative activities before and after thermal treatment of the enzyme. The relative activity in hydrolysis, b, is determined in aqueous medium by the hydrolysis of 2-hydroxy-4-p-nitrophenoxy-butyl decanoate at 80° C. and a certain time like 40 min after and before treatment of the esterase at 100° C. for 40 h in aqueous solution. The relative activity in synthesis, c, is determined in organic medium by the transesterification reaction:
C₁₁H₂₃CO₂CH₃+C₁₀H₂₁OH→C₁₁H₂₃CO₂C₁₀H₂₁+CH₃OH
at 80° C. for a certain period of time, e.g. 24 h, after and before treatment of the enzyme for 24 h at 80° C. in methylcyclohexane. The reaction is followed by gas chromatographic determination of the formed decyl laurate. The value for index a is ≧0.02, preferably ≧0.04, more preferably ≧0.1.

Preferred esterases are lipases. Even more preferred esterases are isolated from the genera Pyrococcus and Thermococcus. Especially preferred is the esterase from a Pyrococcus genus with the amino sequence No. 1.

Amino acid sequence No. 1, Pyrococcus:

| | |
|---|---|
| MIFKAKFGEP KRGWVVIVHG LGEHSGRYAK LVEMLVERGF AVYTFDWPGH GKSSGKRGHT | 60 |
| SVEEAMEIID EIIEEIGEKP FLFGHSLGGL TVIRYAETRP EKVKGVIASS PALAKSPNTP | 120 |
| GFLVALAKFL GVVAPGITFS NGINPNLLSR NKDAVRRYVE DPLVHDKITA KLGRSIFMNM | 180 |
| ELAHREAEKI KVPLLLLVGT QDVITPPEGA RKLFEKLKVE DKEIREFEGA YHEIFEDPEW | 240 |
| GEEFHRVIVE WLEKHS | 256 |

Also especially preferred is the esterase from a Thermococcus genus with the amino acid sequence No. 2.

Amino acid sequence No. 2, Thermococcus:

| | |
|---|---|
| MEVYKVRFGT PERGWVVLVH GLGEHSGRYG RLIKLLNENG FGVYAFDWPG HGKSPGKRGH | 60 |
| TSVEGAMEII DSIIEELGEN PFLFGHSLGG LTVIRYAEAR PDKIRGVIAS SPALAKSPET | 120 |
| PDFMVALAKF LGRIAPGLTL SNGIKPELLS RNRDAVRRYV EDPLVHDRIS AKLGRSIFVN | 180 |
| MDLAHREAEN IRVPILLLVG TGDVITPPKG AKDLFKKLKV EDKELKEFPG AYHEIFEDPE | 240 |
| WGEEFHKTIV EWLLQHSEEG | 260 |

In one embodiment, the procedure of the present invention relates to the production of esters from carboxylic acids, e.g., saturated or unsaturated fatty acids, or carboxylic acid esters, e.g., fatty acid esters, and alcohols, wherein the raw materials are reacted in the presence of the enzyme of the invention, and wherein the formed alcohol or water is removed by distillation or other means like absorption or diffusion.

The process of present invention is performed at temperatures ≧70° C. Temperatures of 70° C. and higher are required to make sure that the reaction mixture is in a liquid state and the viscosity is sufficiently low. Moreover, a minimum temperature often needs to be maintained to facilitate the reaction of immiscible raw materials. The reaction mixture is either a homogenous solution or an emulsion or suspension. The temperature will generally be in the range of 70 to 120° C., preferably 75 to 115° C., more preferably 80 to 110° C. and most preferably 90 to 105° C. The reaction can take place with, or without, solvents, in aqueous, polar or organic media. The reaction time is dependent on the amount of enzyme catalyst used, usually being in the range of 1 to 24 h, preferably between 5 and 10 h. The amount of catalyst will be chosen in adaptation to the activity of the biocatalyst preparation, usually a quantity in the range of 10,000 esterase units (EU) to 2,000,000 EU preferably 20,000 EU to 1,000,000 EU per kg reaction mixture is used.

One EU is the amount of enzyme which hydrolyses one micromole of ester per minute or more generally transforms one micromole of substrate per minute.

The present invention will be further described with reference to the following examples, however, it is to be understood that the present invention is not limited to such examples.

### EXAMPLE 1

### General Methods for Determination of the Esterase Activity

1.1: Hydrolytic Activity

The specific activity of an esterase was expressed in esterase units (EU) per milligramme of biocatalyst:

$$specific activity = \frac{EU}{{mg}_{biocatalyst}} = \frac{\frac{μ {mol}_{reacted substrate}}{min}}{{mg}_{biocatalyst}}$$

The specific activity of the esterase in hydrolysis was measured according to a published method described in D. Lagarde, H. K Nguyen, G. Ravot, D. Wahler, J.-L. Reymond, G. Hills, T. Veit, F. Lefevre, Org. Process Res. Dev., 6 (2002) 441 by monitoring the concentration of nitrophenol liberated from 2-hydroxy-4-p-nitrophenoxy-butyl decanoate (C10-HpNPB) at a wavelength of λ=414 nm. The procedure was as follows: All reagents and buffers were prepared in deionized MilliQ® water. A 20 mM stock solution of C10-HpNPB in acetonitrile was prepared. A BSA solution was prepared as a stock solution (50 mg/ml) in water. A NaIO₄ solution was freshly prepared as a 100 mM stock solution in water. 8 μl of C10-HpNPB stock solution were added to 74 μl of 200 mM PIPES buffer at pH 7.0. The reaction was initiated by adding 10 μl of the enzyme sample. The reaction mixture was incubated at 90° C. for 40 min. The sample was cooled down on ice and BSA (2 mM), NaIO₄, (28 mM) and Na₂CO₃ (40 mM) were added to the mixture. After 10 min of incubation at 25° C. the sample was centrifuged at 6000 g for 5 min and transferred to a microplate. The optical density of the yellow p-nitrophenol was recorded at λ=414 nm using a Spectramax 190 microplate spectrophotometer (Molecular Devices).

For relative activity measurements, the absolute activities were set in relation to each other in percent. For measurement of the absolute activity, the procedure was as follows:

With the known extinction coefficient of p-nitrophenol

$$( {ɛ}_{414 nm}^{pH 7.0} = 14200 {M}^{- 1} \cdot {cm}^{- 1} )$$

the change in p-nitrophenol concentration Δc_{liberated p-nitrophenol} can be calculated with the law of Lambert-Beer

$$Δ {c}_{liberated p - nitrophenol} = \frac{Δ E}{ɛ \cdot d}$$

in which d stands for the layer thickness and ΔE describes the change in extinction. The volumetric activity (vol. activity) is then given by the time-dependent change in concentration of p-nitrophenol

$$vol . activity = \frac{Δ {c}_{liberated p - nitrophenol}}{{t}_{mon}} \cdot {f}_{d}$$
in which tₘₒₙ stands for the monitoring time and f_{d} is the dilution coefficient of the enzyme extract. Finally, the specific activity was calculated by dividing the volumetric activity by the concentration c_{enzyme} of the enzyme containing material (e.g., raw extract, immobilzsed enzyme, pure enzyme)

$$spec . activity = \frac{vol . activity}{{c}_{enzyme}}$$

C10-HpNPB was synthesized from 2-bromo-butene as described for the corresponding fluorescent umbelliferone derivatives (F. Badalassi, D. Wahler, G. Klein, P. Crotti, J.-L. Reymond, Angew. Chem. Int. Ed 39 (2000) 4067; D. Wahler, F. Badalassi P. Crotti J.-L Reymond, Angew. Chem. Int Ed. 40 (2001) 4457).

1.2: Esterification Activity

Activity in esterification was measured by determination of conversion by acid value titration. The conversion (t) for a given point of time t was calculated as follows

$$conversion ( t ) = \frac{acid value ( t = 0 ) - acid value ( t )}{acid value ( t = 0 )}$$

where acid value(t=0) is the initial acid value and acid value(t) is the acid value at the given time t Conversion (t) was plotted against time t and the resulting data points were fitted with the program GraFit (Erithacus Software Ltd., P.O. Box 274, Horley, Surrey, RH69YJ, UK) to an adapted Michaelis-Menten equation

$$conversion ( t ) = {conversion}_{max} \cdot \frac{t}{const + t}$$

In this equation conversionₘₐₓ indicates the maximal conversion and const is a variable describing the bending of the curve. Differentiation for t for calculating the initial slope of the measured curve yields

$$\frac{ⅆ ( conversion )}{ⅆ t} = {conversion}_{max} \cdot \frac{const}{{( const + t )}^{2}}$$

Initial specific enzyme activity k was calculated with t=1 min as follows

$$k = \frac{\frac{ⅆ ( conversion )}{ⅆ t} \cdot μ {moles}_{formed ester}}{{mass}_{biocatalyst} ( mg )}$$

1.3: Transesterification Activity

Transesterification activity was calculated using the mathematical means described in example 1.2 and by determination of conversion (t) with GC. The reaction mixture was derivatized with N-methyl-N-trimethylsilyltrifluoracetamide (MSTFA) and composition of the sample was determined by using a 30 m/0.32 mm apolar capillar GC column with split injection and FID-detection. Conversion(t) at a given point of time t was calculated as follows

$$conversion ( t ) = \frac{{A}_{ester} ( t = 0 ) - {A}_{ester} ( t )}{{A}_{ester} ( t = 0 )}$$

In this Aₑₛₜₑᵣ(t=0) indicates the initial GC surface of the educt ester before reaction and Aₑₛₜₑᵣ (t) presents the GC surface of the educt ester at a given point of time t.

### EXAMPLE 2

### Determination of the Activity Index a

The activity index a is defined as the product
a=b·c
wherein b is the hydrolytic activity fraction and c is the transesterification activity fraction determined by the following methods.
2.1 Hydrolytic Activity Fraction b

The hydrolytic activity fraction b₁ of the esterase before heat treatment and the hydrolytic activity fraction b₂ of the esterase after heat treatment for 40 h at 100° C. were determined by following the change of p-nitrophenol concentration Δc_{liberated p-nitrophenol} due to C10-HpNPB hydrolysis (D. Lagarde, H. K Nguyen, G. Ravot, D. Wahler, J.-L. Reymond, G. Hills, T. Veit, F. Lefevre, Org. Process Res. Dev., 6 (2002) 441) at 80° C. C10-HpNPB was incubated at 80° C. for t=40 min with the enzyme (free or immobilized) and the hydrolytic activity was determined as described in example 1.1. The hydrolytic activity fractions b₁ and b₂ were then calculated according to example 1.1 by the following equation

$${b}_{x} = \frac{volumetric activity}{enzyme concentration}$$

in which the index x is 1 or 2. The hydrolytic activity fraction b is then given by

$$b = \frac{{b}_{2}}{{b}_{1}}$$

2.2 Transesterification Activity Fraction c

The transesterification activity c₁ of the esterase before heat treatment and the transesterification activity c₂ of the esterase after heat treatment for 24 h at 80° C. in methylcyclohexane were determined in the transesterification reaction
methyl laurate+decanol→decyl laurate+methanol
at 80° C. Transesterification activity was calculated similar to the mathematical means from example 1.3. The transesterification activity fraction c is then defined as

$$c = \frac{{c}_{2}}{{c}_{1}}$$

### EXAMPLE 3

### Production of the Free Pyrococcus Esterase

In the following, the esterase is called Est P 1021.

3.1: Culture Medium M21 and Growth Conditions of Pyrococcus Strain Est P 1021

The medium M21 containing (L-1) was prepared as follows:
Yeast Extract 2 g
Casein enzymatic hydrolysate (Sigma P 1192) 4 g
Sea salts 30 g
Cysteine 0.5 g
PIPES 6.05 g
Resazurine (0.1% w/v) 1 ml
Sulphur 10 g

The pH was adjusted to 7.5 with NaOH, the medium was heated to 100° C., cooled and dispensed under N₂/CO₂ (80/20). Before use the medium was reduced with 2 ml of a sterile anaerobic solution of Na₂S·9H₂O (2% W/v) for 100 ml of M21 medium.

3.2: Production of Est P 1021 with the Native Strain

For the production of esterase, 4 anaerobic flasks containing 3 L of medium M21 were prepared. Each flask was inoculated with 120 mL of a fresh over-night culture of Pyrcoccus (P 1021) grown under the conditions described in example 3.1. After 16 h of incubation at 95° C., the culture was centrifuged at 8000 g for 15 min at 4° C. The supernatant was discarded and the cells were resuspended in 27 ml of flesh M21 medium. The cells were lysed by ultrasonification using an amplitude of ultrasonic vibration at the tip of the horn of 10. 10 cycles of 30 sec with 1 min of pause were used (Sonicator ultrasonic liquid processor XL, Misonix Incorporated). Cell debris was removed by centrifugation and proteins were recovered from the supernatant. Protein concentration was measured according to the Bradford assay calibrated against bovine serum albumin (M. M. Bradford, Anal. Biochem. 72 (1976) 248). The specific activity was determined according to example 1.1 to be 5-7 mEU/mg with a stock protein concentration of 8.5-12 mg/ml with the following data:
ΔE=0.682; Δ=14200 M⁻¹ cm⁻¹; d=0.2 cm; tₘₒₙ=40 min; f_{d}=10
   3.3: Cloning of Pyrococcus Esterase (Est P 1021)

A genomic library of the strain P 1021 (7000 clones) has been constructed and screened at 95° C. using C10-HpNPB substrate as previously reported (D. Lagarde, H. K Nguyen, G. Ravot, D. Wahler, J.-L Reymond, G. Hills, T. Veit, F. Lefevre, Org. Process Res. Dev., 6 (2002) 441). Three different clones showing an esterase activity at 95° C. were sequenced. Each clone showed a common open reading frame of 771 bp encoding for the esterase activity. One of this genomic fragments has been directly used for constructing a transformation vector.

3.4: Construction of the Vector pARA P 1021

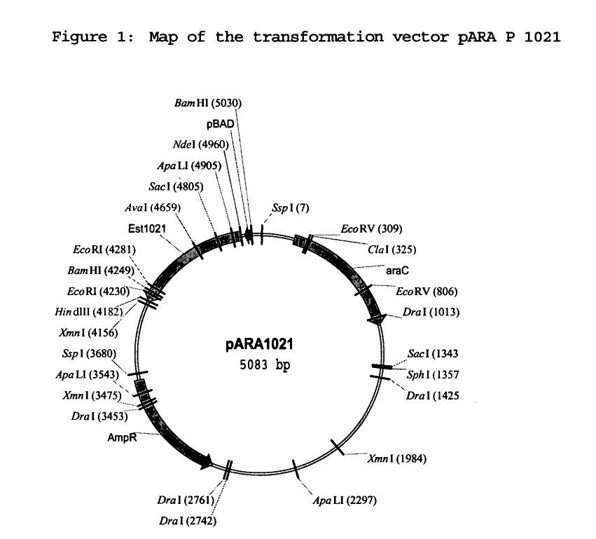

The open reading frame identified as the esterase gene P 1021 was subcloned into a pARA 14 based vector (C. Cagnon, V. Valverde, J. M. Masson: Protein Engineering 4 (1991) 843) in which the NcoI cloning site has been replaced by a NdeI cloning site. A polymerase chain reaction product of the open reading frame amplified from the genomic DNA of Pyrococcus strain P 1021 was obtained using 2 primers carraing a NdeI site in 5′ position and a Hind III site in 3′ position for the cloning of the esterase gene in the pARA based vector under the control of an arabinose inducible promoter. The map of the resulting vector is shown in FIG. 1.

3.5: Production of Recombinant EST P 1021 using the Vector pARA P 1021

The pARA P 1021 vector from example 3.4 was used to transform the E. coli strain MC 1061 pRIL. A 4 litre Erlenmeyer flask fermentation was run in standard Luria Broth (LB) medium containing 10 g/l bactotryptone, 5 g/l yeast extract and 5 g/l sodium chloride. LB medium was supplemented with 100 mg/l ampicillin and 30 mg/l chloramphenicol. The medium was inoculated with 3% v/v of a preculture at 37° C. and pH 7.0. The culture was incubated under shaking (200 rpm). Expression of the esterase gene was induced by addition of 0.02% (v/v) L-arabinose at an optical density at 600 nm of 0.4. Cells were centrifuged 3 h after induction to a final optical density at 600 nm of 1.9. For lysis cells were resuspended in 0.20 M phosphate buffer of pH 8.0 to give a final volume of 30 ml. The cell suspension was then passed once through a high pressure homogeniser at 2 kbar and debris was removed by centrifugation at 13000 g for 20 min. The stock protein concentration was about 20 to 30 mg/ml. The specific activity of the unpurified recombinant raw esterase was determined according to example 1.1 to be 2-3 EU/mg with the following data:
ΔE=0.710; ε=14200 M⁻¹ cm⁻¹; d=0.2 cm; tₘₒₙ=40 min; f_{d}=10000
   3.6: Preparation of Immobilised Recombinant Est P 1021

A culture of recombinant E coli was obtained as described in Example 3.5. The cells were collected by centrifugation at 6000 g for 15 min. The cells were resuspended in 0.25 M Na₂HPO₄/NaH₂PO₄ buffer (pH 8.5) to reach roughly 30-40 g/l of dried cells. Cell disruption was performed with a high pressure homogeniser at 2 kbar. Cell debris was removed by centrifugation and proteins (concentration 15-30 g/l) were recovered from the supernatant. The pH of the mixture was adjusted to 8-8.5 with 0.25 M of Na₂HPO₄/NaH₂PO₄ buffer pH 8.5. 20% of maltitol (w/w) and 10% of glutaraldehyde (w/w) based on the dry weight of the protein were added to the mixture. The suspension was stirred at room temperature for 30 min and 20% of polyazetidine (w/w) based on the dry weight of the protein were added. The reaction mixture was stirred for 15 min at room temperature. The obtained paste was dried overnight at 50° C. The dry pellet was ground to obtain a fine powder.

### EXAMPLE 4

### Characterization of Free Est P 1021

4.1 Activity Index a

The hydrolytic activity fractions b₁ and b₂ were determined according to example 2.1 with non-purified enzyme and the results were as follows:

$${b}_{1} = 6 \frac{mEU}{mg} ; {b}_{2} = 1 \frac{mEU}{mg} ; b = 0.17$$

The transesterification activity fractions c₁ and c₂ were measured according to example 2.2 with freeze-dried, non-purified enzyme as follows:

$${c}_{1} = 2.9 \frac{mEU}{mg} ; {c}_{2} = 0.6 \frac{mEU}{mg} ; c = 0.21$$

and a=0.17·0.21=0.036

4.2 Effect of Temperature on Free Est P 1021 Activity

TABLE 1

| Temperature (° C.) | Relative activity (%) |
|---|---|
| 60 | 46 |
| 70 | 56 |
| 80 | 90 |
| 90 | 96 |
| 95 | 100 |
| 100 | 100 |
| 105 | 93 |

The activity of Est P 1021 was measured by the assay on hydrolytic activity as described in example 1.1 except that temperature was varied. Incubation of Est P 1021 was done in 0.2 M PIPES buffer at pH 7.0. Results are shown above (table 1) with an activity at 100° C. taken as 100% for the esterase of the invention

4.3: Effect of pH on Free Est P 1021 Activity

The activity of Est P 1021 was measured by the assay on hydrolysis activity as described in example 1.1 except that the pH was varied by using different aqueous buffers. Results are shown below with an activity at pH 6 taken as 100%.

TABLE 2

| pH | Relative activity (%) |
|---|---|
| 3 | 19 |
| 5 | 61 |
| 6 | 100 |
| 7 | 38 |
| 8.8 | 10 |

Thermostability of free Est P 1021

4.4

TABLE 3

| Incubation time (hours) | Residual activity (%) |
|---|---|
| 16 | 32 |
| 23 | 32 |
| 40 | 17 |

Samples of culture broth prepared as in Example 3.5 were heat-treated at 100° C. in aqueous solution buffered with 0.2 M PIPES for variable incubation times. Esterase activity of the heat-treated samples was measured using the hydrolysis assay as described in example 1.1. The results are expressed as relative activities compared with a not heat-treated control sample and are shown in table 3.

4.5: Substrate Specificity of Free Est P 1021

The activity of Est P 1021 was measured by the assay on hydrolytic activity as described in example 1.1 except that instead of only C10-HpNPB also the following substrates were test in aqueous solution buffered with 0.2 M PIPES:
2-hydroxy-4-p-nitroxphenoxy-butyl-acetate (C2-HpNPB),
2-hydroxy-4-p-nitrophenoxy-butyl-propionate (C3-HpNPB),
2-hydroxy-4-p-nitrophenoxy-butyl-palmitat (C16-HpNPB),
2-hydroxy-4-p-nitrophenoxy-butyl-stearate (C18-HpNPB),
2-hydroxy-4-p-nitrophenoxy-butyl-oleate (C18′-HpNPB).

The results are given in table 4 with activity for C10-HpNPB taken as 100%.

TABLE 4

| Substrate | Relative activity (%) |
|---|---|
| C2-HpNPB | 27 |
| C3-HpNPB | 100 |
| C10-HpNPB | 100 |
| C16-HpNPB | 79 |
| C18-HpNPB | 52 |
| C18′-HpNPB | 68 |

### EXAMPLE 5

### Characterization of Immobilized Est P 1021

5.1: Activity Index a

The hydrolytic activity fractions b₁ and b₂ were determined according to example 2.1 with enzyme immobilized according to example 3.6 and the results were as follows:

$${b}_{1} = 60 \frac{mEU}{mg} ; {b}_{2} = 36 \frac{mEU}{mg} ; b = 0.6$$

The transesterification activity fractions c₁ and c₂ were measured according to example 2.2 with enzyme immobilized according to example 3.6 as follows:

$${c}_{1} = 52 \frac{mEU}{mg} ; {c}_{2} = 34 \frac{mEU}{mg} ; c = 0.65$$

$$and a = 0.6 \cdot 0.65 = 0.39$$

5.2: Effect of Temperature on Activity of Immobilized Est P 1021 in Water

4 g ground immobilized Est P 1021 were incubated in 60 ml water at 95° C. over a period of 480 min to determine the thermostability of the esterase of the present invention Samples (10 ml) were withdrawn after 120, 240 and 480 min. The water was filtered and the remaining enzyme was dried over night at room tempera in an exsiccator at 60 mbar vacuum. The remaining initial enzyme activity was determined analogous to example 9 with the help of the mathematical means of example 1.2 in n-propyl laurate synthesis and is given as relative activity compared to the non-treated enzyme in table 5. No loss of activity was observed over the period of 480 min.

TABLE 5

| Time (minutes) | Residual activity (%) |
|---|---|
| 120 | 100 |
| 240 | 115 |
| 480 | 95 |

5.3: Effect of pH on Immobilized Est P 1021

The specific hydrolytic activity of immobilised Est P 1021 was measured as described in example 1.1 except that the pH was varied. Results are shown below in table 6 as relative activities, with activity at pH 6 taken as 100%.

TABLE 6

| PH | Relative activity (%) |
|---|---|
| 3 | 95 |
| 4 | 96 |
| 5 | 93 |
| 6 | 100 |
| 7 | 65 |
| 8.8 | 57 |

5.4: Thermostability of Immobilized Est P 1021

Est P 1021 powder (2 mg) prepared as described in example 3.6 was heat-treated at 90° C. and 100° C. for various incubation times. The specific hydrolytic activity of the heat-treated samples and a control sample without heat-treatment were subsequently measured as described in example 1.1. Results are shown in table 7 as relative activities with the activity of the untreated sample taken as 100%.

TABLE 7

Residual
activity (%)

| Incubation time (days) | 90° C. | 100° C. |
|---|---|---|
| 1 | 91 | 80 |
| 2 | 75 | 61 |
| 3 | 55 | 29 |

5.5: Effect of Temperature on Activity of Immobilized Est P 1021 in Organic Solvents

4 g ground immobilized Est P 1021 were incubated in 60 ml methylcyclohexane at 95° C. over a period of 450 min to determine the thermostability of the esterase of the present invention. Samples (10 ml) were withdrawn after 123, 248 and 450 min. The solvent was filtered and the remaining enzyme was dried over night at room temperature in an exsiccator at 60 mbar vacuum. The remaining initial enzyme activity was determined analogously to example 9 with the help of the mathematical means of example 1.2 in n-propyl laurate synthesis and is given as relative activity compared to the non-treated enzyme in table 8. No loss of activity was observed over the period of 450 min.

TABLE 8

| Time | Residual activity (%) |
|---|---|
| 123 | 100 |
| 248 | 85 |
| 450 | 90 |

5.6: Effect of Temperature on Activity of Immobilized Est P 1021 in Water

4 g ground immobilized Est P 1021 were incubated in 60 ml water at 95° C. over a period of 480 mm to determine the thermostability of the esterase of the present invention Samples (10 ml) were withdrawn after 120, 240 and 480 min. The water was filtered and the remaining enzyme was dried over night at room temperature in an exsiccator at 60 mbar vacuum. The remaining initial enzyme activity was determined analogously to example 9 with the help of the mathematical means of example 1.2 in n-propyl laurate synthesis and is given as relative activity compared to the non-treated enzyme in table 9. No loss of activity was observed over the period of 480 min.

TABLE 9

| Time (minutes) | Residual activity (%) |
|---|---|
| 120 | 100 |
| 240 | 115 |
| 480 | 95 |

### EXAMPLE 6

### Production of the Free Thermococcus Esterase

In the following the esterase is called Est P 158.

6.1 Culture Medium M21 and Growth Conditions of Thermococcus Strain P 158

The medium M21 containing (L-1) was prepared as follows:
Yeast Extract 2 g
Casein enzymatic hydrolysate (Sigma P 1192) 4 g
Sea salts 30 g
Cysteine 0.5 g
PIPES 6.05 g
Resazurine (0.1% w/v) 1 ml
Sulphur 10 g

The pH was adjusted to 7.5 with NaOH, the medium was heated to 100° C., cooled and dispensed under N₂/CO₂ (80/20). Before use the medium was reduced with 2 ml of a sterile anaerobic solution of Na₂S·9H₂O (2% w/v) for 100 ml of M21 medium.

6.2 Production of Est P 158 with the Native Thermococcus Strain

For the production of Est P 158, an anaerobic flask containing 2.5 L of medium M21 was prepared. The flask was inoculated with 100 mL of a fresh over-night culture of Thermococcus stain P 158 grown under the conditions described in example 6.1. After 16 h of incubation at 80° C., the culture was centrifuged at 8000 g for 15 min at 4° C. The supernatant was discarded and the cells were resuspended in 12.5 mL of fresh M21 medium. The cells were lysed by ultrasonification. 10 cycles with an amplitude at the tip of the horn of 6, 15 cycles with an amplitude of 7 and 3 cycles with an amplitude of 9 were applied. Each cycle was 30 sec with 1 min of pause (Sonicator ultrasonic liquid processor XL, Misonix Incorporated). Cell debris was removed by centrifugation and proteins were recovered in the supernatant. Protein concentration was measured according to the Bradford assay calibrated against bovine serum albumin (M. M. Bradford, Anal. Biochem. 72 (1976) 248) The specific activity was determined according to example 1.1 to be 8-10 mEU/mg with a stock protein concentration of 4.5-5.5 mg/ml with the following data:
ΔE=0.511; ε=14200 M⁻¹ cm⁻¹; d=0.2 cm; tₘₒₙ=40 min; f_{d}=10
   6.3: Cloning, Production and Immobilization of Est P 158

Cloning, production and immobilization of Est P 158 was performed as described for EstP 1021 in Examples 3.3-3.6.

### EXAMPLE 7

### Characterisation of Free Est P 158

7.1: Activity Index a

The hydrolytic activity fractions b₁ and b₂ were determined according to example 2.1 with partially purified enzyme and the results were as follows:

$${b}_{1} = 16 \frac{EU}{mg} ; {b}_{2} = 2 \frac{EU}{mg} ; b = 0.125$$

The transesterification activity fractions c₁ and c₂ were measured according to example 2.2 with freeze-dried partially purified enzyme as follows:

$${c}_{1} = 2.4 \frac{EU}{mg} ; {c}_{2} = 0.73 \frac{mEU}{mg} ; c = 0.30$$

$$and$$

$$a = 0.125 \cdot 0.30 = 0.038$$

7.2: Effect of Temperature on Esterase Activity of Est P 158

The activity of the Est P 158 was measured by the assay on hydrolytic activity as described in example 1.1 except that temperature was varied. Results are shown below as relative activities (table 10) with an activity at 70° C. taken as 100% for the esterase of the invention.

TABLE 10

| Temperature (° C.) | Residual activity (%) |
|---|---|
| 70 | 100 |
| 80 | 92 |
| 90 | 87 |
| 95 | 75 |

7.3: Thermostability of Free Est P 158

Samples of culture broth prepared as in Example 6.2 were heat-treated at 85° C. in buffered aqueous solution (200 mM PIPES buffer at pH 7.0) for variable incubation times. Est P 158 activity of the heat-treated samples were measured using the hydrolysis assay as described in example 1.1. Results are expressed as relative activities compared to an unincubated Est P 158 sample and are shown in table 11.

TABLE 11

| Incubation time at 85° C. (hours) | Residual activity (%) |
|---|---|
| 1 | 98 |
| 2 | 82 |
| 24 | 57 |

7.4: Substrate Specificity of Free Est P 158

The activity of Est P 158 esterase was measured by the assay on hydrolytic activity as described in example 1.1 except that instead of only C10-HpNPB also the following substrates were tested: (C16-HpNPB), (C18-HpNPB); (C18-oleate-HpNPB). The results are given in table 12 with activity for C10 taken as 100%.

TABLE 12

| Cn-HpNPB substrate | Relative activity (%) |
|---|---|
| C10 | 100 |
| C16 | 77.3 |
| C18 | 24.1 |
| C18 oleate | 65.7 |

### EXAMPLE 8

### Characterization of Immobilised Est P 158

8.1: Activity Index a

The hydrolytic activity fractions b₁ and b₂ were determined according to example 2.1 with partially purified enzyme immobilized according to example 3.6 and the results were as follows:

$${b}_{1} = 25 \frac{EU}{mg} ; {b}_{2} = 15 \frac{EU}{mg} ; b = 0.6$$

The transesterification activity fractions c₁ and c₂ were measured according to example 2.2 with partially purified enzyme immobilized according to example 3.6 as follows:

$${c}_{1} = 48 \frac{EU}{mg} ; {c}_{2} = 25 \frac{mEU}{mg} ; c = 0.52$$

$$and$$

$$a = 0.6 \cdot 0.52 = 0.31$$

8.2: Thermostability of Immobilized Native Est P 158

Esterase powder (about 2 mg) prepared like it is described for Est P 1021 in Example 3.6 was heat-treated at 85° C. for various incubation times in buffered aqueous solution (200 mM PIPES buffer at pH 7.0). The hydrolytic activity of the heat-treated samples were subsequently measured as described in example 1.1. Results are given as relative activities compared to an unincubated immobilised Est P 158 sample and are shown in table 13.

TABLE 13

| Incubation time(day) | Residual activity at 70° C. (%) |
|---|---|
| 1 | 80 |
| 2 | 75 |

### EXAMPLE 9

### Esterification of Propanol and Lauric Acid with Immobilized Est P 1021

TABLE 14

| Time/min | Conversion |
|---|---|
| 0 | 0 |
| 17 | 0.0607 |
| 37 | 0.0401 |
| 124 | 0.0846 |
| 158 | 0.1036 |
| 186 | 0.0849 |
| 236 | 0.0936 |
| 329 | 0.0262 |
| 433 | 0.0432 |
| 1354 | 0.1612 |
| 1530 | 0.1812 |
| 1677 | 0.1836 |
| 1829 | 0.1864 |
| 2866 | 0.2315 |
| 3270 | 0.2480 |
| 4917 | 0.2546 |

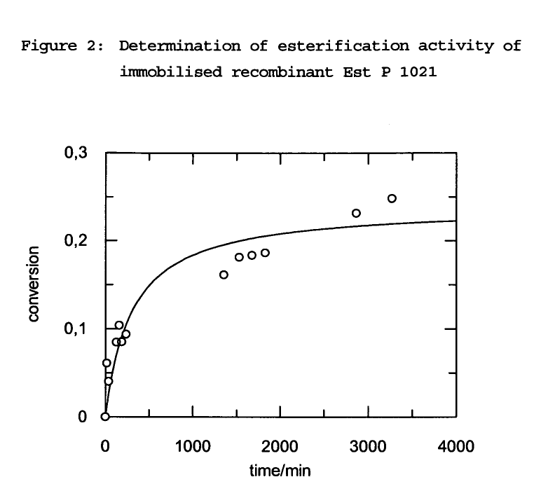

In order to assess the activity of Est P 1021 9.25 g n-propanol and 30.85 g lauric acid were heated up to 60° C. without solvent and 2 g of ground immobilized Est P 1021 prepared as described in example 3.6 were added. Acid value was determined by titration with 0.1 N KOH in ethanol to calculate the conversion of the reaction. The results are given in table 14 and FIG. 2. Esterase synthesis activity in n-propyl laurate synthesis was calculated analogous to example

$$1.2 to be k = 60 \frac{mU}{mg}$$

with the following parameters for the adapted Michaelis-Menten equation: conversionₘₐₓ=0,24; const=303 niun for t=1 min.

### EXAMPLE 10

### Esterification of Myristyl Alcohol and Myristic Acid with Immobilized Est P 1021

34.3 g myristic acid and 32.2 g myristyl alcohol were heated to 95° C. and 4 g ground esterase according to the invention were added. Samples (10 g) of the reaction mixture were withdrawn after 1.96, 3.9, and 7.72 days and mixed with 40 g warm acetone, filtered and washed again with 20 g warm acetone twice to remove the ester. After drying over night in an exsiccator under 20 mbar vacuum and ambient temperature the initial activity was determined analogously to example 9 with the help of the mathematical means of example 1.2 in n-propyl laurate synthesis and is expressed as relative activity compared to the non-treated enzyme. No loss of activity was recognised over this period of 7.72 days (table 15).

TABLE 15

| Reaction time (days) | Residual activity (%) |
|---|---|
| 1.96 | 100 |
| 3.9 | 90 |
| 7.72 | 95 |

### EXAMPLE 11

### Transesterification of Methyl Laurate and Decanol with Immobilized Esterase Est P 1021

23.7 g decanol and 32.2 g methyl laurate were heated up to 95° C. and 1.4 g of ground immobilized esterase from example 13 were added. Formed methanol was removed at 300 mbar. After 48 h the reaction was stopped and the catalyst was removed from the reaction mixture by filtration. The composition of the reaction mixture and the conversion was determined with gas chromatography (GC) according to example 1.3. Retention times: 3,5 min (decanol); 5,1 min (methyl laurate); 12,7 min (decyl laurate). The conversion was determined to 95%.

### EXAMPLE 12

### Repeated use of the Immobilized Est P 1021 in Myristyl Myristate Synthesis

TABLE 16

| Reuse No. | Time/min | Conversion | $Initial activity / [ \frac{mU}{mg} ]$ |
|---|---|---|---|
| 2 | 0 | 0 | 40 ± 7 |
| | 30 | 0.052 | |
| | 64 | 0.096 | |
| | 128 | 0.136 | |
| | 246 | 0.194 | |
| | 380 | 0.251 | |
| | 430 | 0.267 | |
| | 1400 | 0.501 | |
| 10 | 0 | 0 | 42 ± 5 |
| | 31 | 0.035 | |
| | 62 | 0.059 | |
| | 124 | 0.105 | |
| | 266 | 0.185 | |
| | 380 | 0.235 | |
| | 450 | 0.271 | |
| | 1460 | 0.490 | |

34.3 g myristic acid and 32.2 g myristyl alcohol were heated up to 95° C. and 4 g ground immobilized esterase as prepared in example 3.6 were added. Samples of the reaction mixture were withdrawn after 1, 2, 4, 8 and 24 h for conversion determination by acid value titration. The initial activity in myristyl myristate synthesis was determined by the help of the mathematical means from example 1.2. After 24 h the reaction was stopped and the enzyme was recovered by mixing the ester with 200 g warm acetone, filtering and washing the residual enzyme with 40 g warm acetone twice. After drying the enzyme in an exsiccator over night under 20 mbar vacuum and ambient temperature, the recovered enzyme was used again in myristyl myristate synthesis in the same manner as described above and was shown to maintain its activity following the above described procedure. The procedure was repeated 10 times. The enzyme was stable throughout the entire test, in the last cycle a relative activity of approx. 95% was found which is within the detection limits. The results are shown in table 16.

### EXAMPLE 13

### Esterification of Diglycerol with Caprylic Acid with Immobilze Est P 1021

50 g diglycerol and 43.3 g caprylic acid were heated to 95° C. and 3.7 g of ground immobilized Est P 1021 from example 3.6 were added. Formed water was removed at 50 mbar. After 48 h the reaction was stopped and the catalyst was removed from the reaction mixture by filtration. The acid value was measured by titration with 0.1 N KOH in ethanol and the conversion was calculated to be 89% referred to caprylic acid.

### EXAMPLE 14

### Synthesis of N-stearoyl Stearylamide by Reaction of Methyl Stearate and Stearyl Amine and Immobilized Est P 1021

24.3 g stearyl amine and 26.9 g methyl stearate were heated to 95° C. and 2 g of ground immobilized esterase from example 3.6 were added. After 48 h at 500 mbar the reaction was stopped and the catalyst was removed from the reaction mixture by filtration. The composition of the reaction mixture and the conversion referred to methyl stearate was determined to 95% by GC according to example 1.3. Retention times: 8,6 min (stearyl amine); 9.4 min (methyl stearate); 21.1 min (stearoyl stearylamide).

### EXAMPLE 15

### Synthesis of Ethyleneglycol Dihydroxystearate from Ethyleneglycol and 12-hydroxystearic Acid with Immobilize Est P 1021

324 g 12-hydroxystearic acid and 32.4 g ethyleneglycol were heated to 95° C. and 22 g of ground immobilized Est P 1021 from example 3.6 were added. Formed water was removed at 300 mbar. After 48 h the reaction was stopped and the catalyst was removed from the reaction mixture by filtration. The acid value was measured by titration with 0.1 N KOH in ethanol and the conversion was calculated to be 92% referred to 12-hydroxy-stearic acid.

### EXAMPLE 16

### Synthesis of 4-hydroxybutylstearylamide by Reaction of Methyl Stearate and 4-amino-1-butanol and Immobilized Esterase P 1021

11.6 g 4-amino-1-butanol and 38.8 g methyl stearate were heated to 95° C. and 2 g of ground immobilized ester were added. After 48 h at 500 mbar the reaction was stopped and the catalyst was removed from the reaction mixture by filtration The composition of the reaction mixture and the conversion referred to methyl stearate was determined to be 92% by GC according to example 1.3. Retention times: 8,8 min (4-amino-1-butanol); 9.4 min (methyl stearate); 24.8 min (4-hydroxybutyl-stearylamide).

### EXAMPLE 17

### Esterification of Behenic Acid and 2-ethylhexanol with Immobilizsed Est P 158

34 g behenic acid and 13 g 2-ethylhexanol were heated to 85° C. and 1.9 g of ground immobilized Est P 158 prepared like it is described for Est P 1021 in Example 3.6 were added. Formed water was removed at 50 mbar. After 48 h the reaction was stopped and the catalyst was removed from the reaction mixture by filtration. The acid value was measured by titration with 0.1 N KOH in ethanol and the conversion was calculated to be 84% referred to behenic acid.

### EXAMPLE 18

### Comparative Example of Free Est P 1021, Lipase PL and QL from Alcaligenes and Lipase B from Candida antarctica

Free enzyme solutions (lipases QL and PL 1 mg/ml; lipase B (state of the art) and Est P 1021 (inventive) 0.01 mg/ml) were heat-treated at 90° C. for variable incubation times in an aqueous solution buffered with 0.2 M PIPES. Specific esterase activity of the heat-treated samples was measured using the hydrolysis assay as described in example 1.1., except that the assay temperature for lipases QL and PL was set to 40° C. and that for lipase B to 60° C. At these temperatures, the highest activities for the not heat-treated enzymes were found. Results are expressed as relative activities compared to the not heat-treated enzymes and are given in table 17.

TABLE 17

| | | | | Residual |
|---|---|---|---|---|
| Incubation | Residual | Residual | Residual | activity |
| time | activity of | activity of | activity of | of Est |
| (hours) | lipase QL (%) | lipase PL (%) | lipase B (%) | P 1021 (%) |
| 0 | 100.0 | 100.0 | 100.0 | 100.0 |
| 1 | 0.0 | 64.9 | 3.2 | 92.7 |
| 6 | 0.0 | 30.2 | 0.0 | 89.5 |
| 8 | 0.0 | 21.5 | 0.0 | 86.2 |
| 24 | 0.0 | 8.7 | 0.0 | 56.4 |

### EXAMPLE 19

### Comparative Example of Immobilised Est P 1021 and Immobilised Lipase B from Candida antarctica (Chirazyme L-2)

Esterase powder (2 mg) prepared as described in example 3.6 and Chirazyme L-2 (2 mg, Roche) were heat-treated at 95° C. for one week under continuous stirring in the presence of an equimolar amounts of methyl laurate and decanol.

TABLE 18

| | Biocatalyst | |
|---|---|---|
| | Chirazyme L-2 | Immobilised Est P 1021 |
| Residual activity (%) | 0 | 60 |

Afterwards the biocatalyst was recovered and its transesterification reaction rate was determined by GC according to example 1.3 in the transesterification of methyl laurate with decanol. Chirazyme L-2 lost all its activity under these conditions whereas immobilised Est P 1021 lost only 40% of its initial activity. Results are expressed as relative activities compared to the not heat-treated biocatalysts and are given in table 18. FIG. 1 shows the map of transformation vector pARA P 1021. FIG. 2 shows the determination of esterification activity of immobilized recombinant Est P 1021.

While the present invention has been particularly shown and described with resect to preferred embodiments thereof it will be understood by those skilled in the art that the foregoing and other changes in forms and details may be made without departing from the spirit and scope of the present invention. It is therefore intended that the present invention not be limited to the exact forms and details described and illustrated, but fall within the scope of the appended claims.

What is claimed is:

1. A process for producing an acyl compound having a formula
R¹[(—X—R²)ₙR³]ₚ
wherein R¹ is hydrogen or an organic or silicone-organic residue which can be cyclicly connected to R⁶, X is C(O)—Y, Y—C(O), C(O)—R⁴—C(O)—Y, or Y—C(O)—R⁴—C(O), R² is a group of divalent organic residues containing p members from R^{2.1} to R^{2.P} which can be equal or different, each containing at least one carbon atom, R³ is a chemical univalent link or selected from the group of hydrogen, an hydroxyl group, an alkyl group which can be cyclicly connected to R⁶, the group Y—C(O)—R⁴H, or Y—C(O)—R⁵—C(O)—OH, n is an integer number ≧1, p is an integer number from 1 to 100, Y is O, NR⁶, or S, R⁴ is a divalent hydrocarbon group which can be saturated or unsaturated, linear, branched, or cyclic or a silicone-organic residue, R⁵ is a divalent hydrocarbon group which can be saturated or unsaturated, linear, branched or cyclic, not substituted or substituted by hydroxy, alkoxy, hydroxycarbonyl or alkoxycarbonyl groups, R⁶ is hydrogen, a mono or divalent hydrocarbon group, which can be saturated or unsaturated, linear, branched, or cyclic, not substituted or substituted by hydroxy or alkoxy groups, and cyclicly connected to R¹ or R³, by contacting an immobilized thermostable esterase with carboxylic acid derivatives and water, alcohols, amines, or thiols for hydrolysis or the formation of esters, amides, or thioesters, wherein said esterase
a) retains, in its' free form, at least 10% of its' initial hydrolysis activity after treatment for 40 h at 80° C. in aqueous solution,
b) has an optimal temperature of 70 to 110° C., and
c) is suitable for repeated use in said process at temperatures above 70° C.

2. A process according to claim 1, wherein said erase retains, in its' free form, at least 10% of its initial hydrolysis activity after treatment for 40 h at 90° C. in an aqueous solution.

3. A process according to claim 1 wherein said esterase has an activity index a ≧0.02, wherein a=b·c and b is the fraction of relative activity in the hydrolysis at 80° C. and 40 min. of 2-hydroxy-4-p-nitrophenoxy-butyl decanoate after versus before treatment of the enzyme for 40 h at 100° C. in aqueous solution and c is the fraction of relative activity in the trans-esterification reaction methyl laurare+decanol→decyl laurate+methanol at 80° C. and 24 h after versus before treatment of the enzyme for 24 h at 80° C. in methylcyclohexane.

4. The process according to claim 1, wherein the contacting comprises an esterification, transesterification, or amidation reaction.

5. The process according to claim 1, wherein esters are produced from carboxylic acids or carboxylic acid esters and alcohols.

6. The process according to claim 1, wherein primary or secondary amides are produced from carboxylic acids or carboxylic acid esters and primary or secondary amines.

7. The process according to claim 1, wherein the thermostable esterase is immobilized on a substrate which is monomeric or polymeric.

8. The process according to claim 1, wherein the thermostable esterase is immobilized on a substrate having at least 8 carbon atoms.

9. The process according to claim 1, wherein the thermostable esterase is immobilized on a substrate having 2-50 carbon atoms.

10. The process according to claim 1, wherein the thermostable esterase is immobilized on a substrate that is fat or oil derived

11. The process according to claim 1, wherein the thermostable esterase is immobilized on a substrate that is a silicone derivative.

12. The process according to claim 1, wherein the thermostable esterase is immobilized on a substrate having a molecular weight of 10 to 100,000.

13. The process according to claim 1, wherein the thermostable esterase is immobilized on a substrate which comprises a reaction product between at least one polar and at least one nonpolar substrate.

14. The process according to claim 1, wherein water is involved and the reaction is conducted in a pH range of pH 3-pH 9.

15. The process according to claim 1, wherein the formed water or alcohol are removed during the reaction.

16. The process according to claim 1, further comprising the step of recycling the esterase.

17. The process according to claim 1 wherein the esterase is derived from the genera Pyrococcus or Thermococcus.
